(19) United States
(12) Patent Application Publication
(10) Pub. No.: US 2026/0224846 A1
(43) Pub. Date: Aug. 6, 2026

(54) SYSTEMS AND METHODS FOR CATHETER BALLOON REMOVAL

(71) Applicant: Mavericks Foundation LLC, La Grange, IL (US)

(72) Inventors: Ravi Ramana, La Grange, IL (US); Rahul Sharma, Palo Alto, CA (US)

(73) Assignee: Mavericks Foundation LLC, La Grange, IL (US)

(21) Appl. No.: 19/151,436

(22) PCT Filed: Jan. 24, 2024
(86) PCT No.: PCT/US2024/012742

§ 371 Date: Jul. 28, 2025

Related U.S. Application Data
(60) Provisional application No. 63/482,004, filed on Jan. 27, 2023.

(51) Int. Cl. A61M 25/00; A61B 17/34; A61M 25/06; A61M 25/09; A61M 25/10

(52) U.S. Cl. CPC A61M 25/0068; A61B 2017/3425; A61B 2017/3486; A61M 2025/0681; A61M 25/09; A61M 2025/1061; A61M 2025/1081; A61M 2025/1084

(57) ABSTRACT

Balloon catheter systems and methods of use are provided. The balloon catheter system may comprise an elongated catheter shaft, having a proximal end and a distal end, enclosing a guidewire, a balloon system, and a balloon retrieval system. The balloon catheter system may comprise the guidewire, the balloon system, comprising a balloon positioned along the guidewire, and the balloon retrieval system. The balloon retrieval system may comprise a flowering portion configured to expand from the distal end of the elongated catheter shaft, and a sheath portion positioned within the elongated catheter shaft. The flowering portion may be configured to funnel the balloon, in a deflated configuration, into the sheath portion, and the sheath portion may be configured to enable the balloon, in the deflated configuration, to be pulled through a central cavity therethrough.

### CROSS-REFERENCE TO RELATED APPLICATIONS

This application claims priority to U.S. Provisional Patent Application No. 63/482,004, filed Jan. 27, 2023, the contents of which are incorporated herein by reference in its entirety.

### TECHNICAL FIELD

Embodiments of the present disclosure relate to balloon catheter systems and methods of use and, in particular, to balloon catheter systems having balloon retrieval systems.

### BACKGROUND

Medical catheters are often used during treatment of cardiovascular disorders. These disorders often require patients to undergo invasive cardiovascular procedures, which can accompany the use of medical catheters.

Due to the delicate nature of the cardiovascular system, the use of medical catheters in the cardiovascular system are invasive and potentially dangerous procedures (e.g., percutaneous structural heart interventions). Due to the high stakes of such procedures, it is imperative that medical catheters be configured for accuracy and reproducibility. The greater the accuracy and reproducibility, the greater the chances of improvement in short-and long-term clinical outcomes of cardiovascular patients.

Catheters used during cardiovascular procedures often incorporate the use of one or more balloons. These balloons are often passed through the catheter and placed into position within a patient before inflation. These balloons are often highly compacted prior to inflation to facilitate movement through the catheter. However, after inflation and subsequent deflation, these balloons are no longer highly compacted, hindering removal of the balloons through the catheter during extraction of the balloons.

For at least these reasons, systems and methods for removing catheter balloons after deployment of the balloons is needed.

### SUMMARY

According to an aspect of the present disclosure, a balloon catheter system is provided. The balloon catheter system may comprise an elongated catheter shaft, having a proximal end and a distal end, enclosing a guidewire, a balloon system, and a balloon retrieval system. The balloon catheter system may comprise the guidewire, the balloon system, comprising a balloon positioned along the guidewire, and the balloon retrieval system. The balloon retrieval system may comprise a flowering portion configured to expand from the distal end of the elongated catheter shaft, and a sheath portion positioned within the elongated catheter shaft. The flowering portion may be configured to funnel the balloon, in a deflated configuration, into the sheath portion, and the sheath portion may be configured to enable the balloon, in the deflated configuration, to be pulled through a central cavity therethrough.

According to an exemplary embodiment, the elongated catheter shaft may be a 14 French (Fr) catheter shaft.

According to an exemplary embodiment, the elongated catheter shaft may be configured to enclose a plurality of lumens, comprising the flowering portion and the sheath portion of the balloon retrieval system, and a balloon inflation lumen configured to inflate and deflate the balloon.

According to an exemplary embodiment, the balloon system may comprise the balloon inflation lumen, and the balloon may be configured to be inflated exterior to the distal end of the elongated catheter shaft.

According to an exemplary embodiment, the sheath portion may be configured to carry the balloon through the elongated catheter shaft prior to inflation of the balloon.

According to an exemplary embodiment, the balloon system may comprise a balloon inflation port configured to supply at least one inflation medium through the balloon inflation lumen to the balloon.

According to an exemplary embodiment, the flowering portion may be configured to retract after expansion in order to be pulled through the elongated catheter shaft.

According to an exemplary embodiment, the sheath portion may have a diameter of 10-15 Fr.

According to an aspect of the present disclosure, a method of retrieving a catheter balloon is provided. The method may comprise inserting a balloon through a balloon catheter system. The balloon catheter system may comprise an elongated catheter shaft, having a proximal end and a distal end, enclosing a guidewire, a balloon system, and a balloon retrieval system. The balloon catheter system may comprise the guidewire, the balloon system, comprising a balloon positioned along the guidewire, and the balloon retrieval system, comprising a flowering portion configured to expand from the distal end of the elongated catheter shaft, and a sheath portion positioned within the elongated catheter shaft. The flowering portion may be configured to funnel the balloon, in a deflated configuration, into the sheath portion, and the sheath portion may be configured to enable the balloon, in the deflated configuration, to be pulled through a central cavity therethrough. Inserting the balloon through the balloon catheter system may comprise inserting the balloon through the sheath portion beyond the distal portion of the elongated catheter shaft. The method may comprise inflating the balloon exterior to the distal end of the elongated catheter shaft, deflating the balloon, expanding the flowering portion, and pulling the balloon, in a deflated configuration, through the flowering portion and through the sheath portion, thereby pulling the balloon through the elongated catheter shaft.

According to an exemplary embodiment, the elongated catheter shaft may be a 14 French catheter shaft.

According to an exemplary embodiment, the elongated catheter shaft may be configured to enclose a plurality of lumens, comprising the flowering portion and the sheath portion of the balloon retrieval system, and a balloon inflation lumen configured to inflate and deflate the balloon.

According to an exemplary embodiment, the balloon system may comprise the balloon inflation lumen and a balloon inflation port configured to supply at least one inflation medium through the balloon inflation lumen to the balloon. According to an exemplary embodiment, the inflating the balloon may comprise supplying the at least one inflation medium through the balloon inflation lumen to the balloon.

According to an exemplary embodiment, the method may further comprise, after pulling the balloon, in the deflated configuration, through the flowering portion and through the sheath portion, retracting the flowering portion and pulling the flowering portion through the elongated catheter shaft.

According to an exemplary embodiment, the sheath portion may have a diameter of 10-15 Fr.

### BRIEF DESCRIPTION OF THE DRAWINGS

The accompanying drawings, which are incorporated herein and constitute part of this specification, illustrate the presently preferred embodiments of the present disclosure, and, together with the general description above and the detailed description given below, serve to explain the features of the present disclosure. In the drawings:

FIG. 1 is a side view of a balloon catheter system having a balloon retrieval system in a contracted position, according to an exemplary embodiment of the present disclosure;

FIG. 2 is a side view of a balloon catheter system having a balloon retrieval system in an expanded position, according to an exemplary embodiment of the present disclosure; and

FIG. 3 is a flow chart of a method for using a balloon catheter system having a balloon retrieval system, according to an embodiment of the present disclosure.

### DETAILED DESCRIPTION

Embodiments of the present disclosure will now be described more fully hereinafter. However, many modifications and other embodiments of the present disclosure set forth herein will come to mind to one skilled in the art to which the present disclosure pertains having the benefit of the teachings presented in the foregoing descriptions. Therefore, it is to be understood that the present disclosure is not to be limited to the specific embodiments disclosed and that modifications and other embodiments are intended to be included within the scope of the appended claims.

Reference throughout this specification to features, advantages, or similar language does not imply that all of the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout the specification may, but do not necessarily, refer to the same embodiment.

Furthermore, the described features, advantages, and characteristics of the present disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the disclosure.

Reference throughout this specification to “one embodiment,” “an embodiment,” or similar language means that a particular feature, structure, or characteristic described in connection with the indicated embodiment is included in at least one embodiment of the present disclosure. Thus, the phrases “in one embodiment,” “in an embodiment,” “in some embodiments,” and similar language throughout this specification may, but do not necessarily, all refer to the same embodiment.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to.”

When used in this document, the term “exemplary” is intended to mean “by way of example” and is not intended to indicate that a particular exemplary item is preferred or required.

As used herein, the terms “about” and “approximately” may be used interchangeably and is meant to encompass variations of ±20%, ±10%, +5%, ±1%, and ±0.1% from the specified value, as such variations are appropriate.

As used herein, the term “communicate” and “communication” include, but are not limited to, the connection of fluid system elements, either directly or remotely, enabling fluid interface among and between said elements.

As used herein, the term “connectable” or “connection” refers to being able to be joined together for purposes including, but not limited to, allowing a flow of fluid. The term “connectable” can refer to being able to be joined together temporarily or permanently.

As used herein, a “patient” or “subject” is a member of any animal species, preferably a mammalian species, optionally a human. The subject can be an apparently healthy individual, an individual suffering from a disease, or an individual being treated for a disease.

As used herein, the terms “treating” and “treatment” refer to the management and care of a patient having a pathology or condition by administration of one or more therapies contemplated by the present disclosure. Treating also includes administering one or more methods of the present disclosure or using any of the systems, devices, or compositions of the present disclosure in the treatment of a patient. As used herein, “treatment” or “therapy” refers to both therapeutic treatment and prophylactic or preventative measures. “Treating” or “treatment” does not require complete alleviation of signs or symptoms, does not require a cure, and includes protocols having only a marginal or incomplete effect on a patient.

As used herein, the term “vessel” refers to a bodily passage or tract through which a catheter of the present disclosure may be disposed. This may include, e.g., blood vessels, arteries, veins within the circulatory system, the digestive tract, urinary tract, biliary tract, body cavities, or other passages in the body.

The present disclosure relates to balloon catheters having balloon retrieval systems and methods of use. According to various embodiments, the present disclosure provides technology that enables an efficient and reproducible approach to insert a balloon through a catheter, deploy the balloon from a distal end of the catheter, inflate the deployed balloon, deflate the deployed balloon, and extract the balloon through the catheter via a balloon retrieval system. This further aids in improving clinical outcomes for patients (e.g., the systems and methods aid in preventing deployed balloons from becoming lodged, or temporarily lodged, within catheters during removal of the balloons, aids in decreasing the time needed to remove the balloons, etc.).

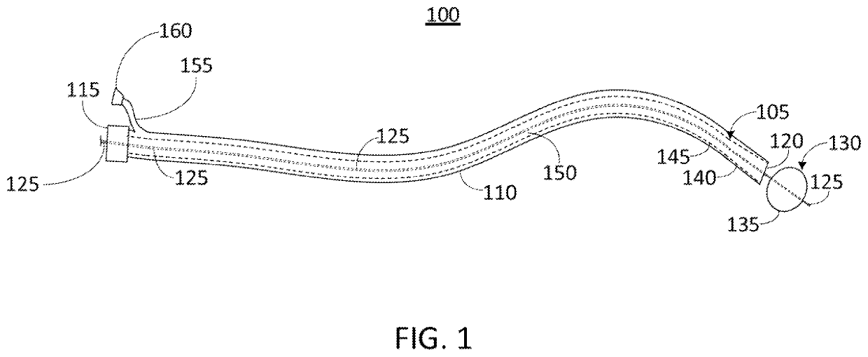

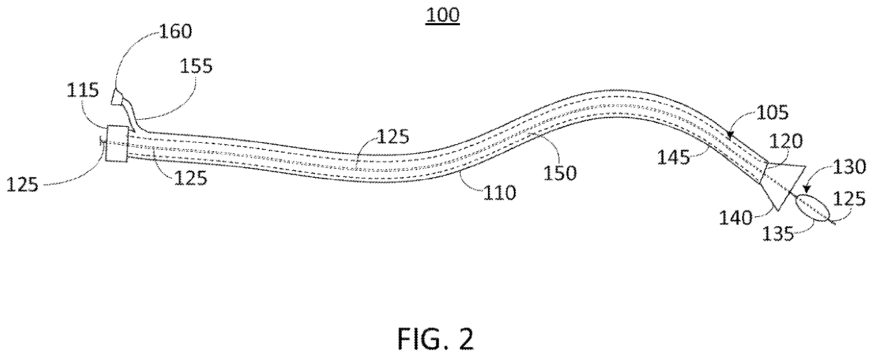

Referring now to FIGS. 1-2, a side view of a balloon catheter system 100 having a balloon retrieval system 105 in a contracted position (FIG. 1) and a balloon retrieval system 105 in an expanded position (FIG. 2) are illustratively depicted, in accordance with various embodiments of the present disclosure.

According to various embodiments, the balloon catheter system 100 comprises an elongated catheter shaft 110 having a proximal end 115 and a distal end 120. According to various embodiments, the elongated catheter shaft 110 may be configured to enclose a guidewire 125, a balloon system 130, and the balloon retrieval system 105.

According to various embodiments, the balloon system 130 may comprise a balloon 135, e.g., a catheter balloon used in conjunction with a catheter. The balloon 135 may be positioned along the guidewire 125. According to various embodiments, the balloon 135 may surround all or a portion of a circumference of the guidewire 125 (enabling the balloon 135 to be introduced over the guidewire 125) and/or may be configured to be moved in conjunction with movement of the guidewire 125. According to various embodiments, the balloon 135 may be coupled to the guidewire 125. The balloon 135 may be a vascular balloon and/or other suitable balloon.

According to various embodiments, the balloon retrieval system 105 may comprise a flowering portion 140, configured to expand from the distal end 120 of the elongated catheter shaft 110, and a sheath portion 145, positioned within the elongated catheter shaft 110. The sheath portion 145 may be a vascular sheath. According to various embodiments, the flowering portion 140 may be configured to funnel the balloon 135, in a deflated configuration, into the sheath portion 145. The sheath portion 145 may be configured to enable the balloon 135, in the deflated configuration, to be pulled through a central cavity 150 therethrough. According to various embodiments, the sheath portion 145 may be configured to carry the balloon 135 through the elongated catheter shaft 110 prior to inflation of the balloon 135. According to various embodiments, the sheath portion 145 may have a diameter of approximately 10-15 French (Fr).

According to various embodiments, the elongated catheter shaft 110 may be an approximately 14 Fr catheter shaft. It is noted, however, that elongated catheter shaft 110 may have other suitable dimensions which may be incorporated, while maintaining the spirit and functionality of the present disclosure. The elongated catheter shaft 110 may be configured to enclose a plurality of lumens. The plurality of lumens may comprise, e.g., the flowering portion 140 and the sheath portion 145 of the balloon retrieval system 105, a balloon inflation lumen 155 configured to inflate and/or deflate the balloon 135, and/or one or more other suitable lumens. The balloon system 130 may comprise the balloon inflation lumen 155 which may be configured to transport at least one inflation medium to the balloon 135. According to various embodiments, the balloon catheter system 100 may comprise a balloon inflation/deflation port 160 configured to supply the at least one inflation medium through the balloon inflation lumen 155 to the balloon 135 for inflating the balloon 135. According to various embodiments, the balloon 135 may be configured to be inflated exterior to the distal end 120 of the elongated catheter shaft 110.

As shown, e.g., in FIG. 2, the balloon retrieval system 105 may be configured to be moved along the elongated catheter shaft 110, causing the flowering portion 140 to extend past the distal end 120 of the elongated catheter shaft 110. According to various embodiments, once extended past the distal end 120 of the elongated catheter shaft 110, the flowering portion 140 may be configured to expand in shape, forming a funnel which opens distally from the distal end 120 of the elongated catheter shaft 110. According to various embodiments, this funneling may be configured to enable the flowering portion 140 to enable safe, reliable withdrawal of larger bore vascular balloons 135 into smaller, low profile, vascular sheaths 145.

According to various embodiments, the flowering portion 140 may be configured to retract after expansion in order to enable the balloon retrieval system 105 to be pulled through the elongated catheter shaft 110.

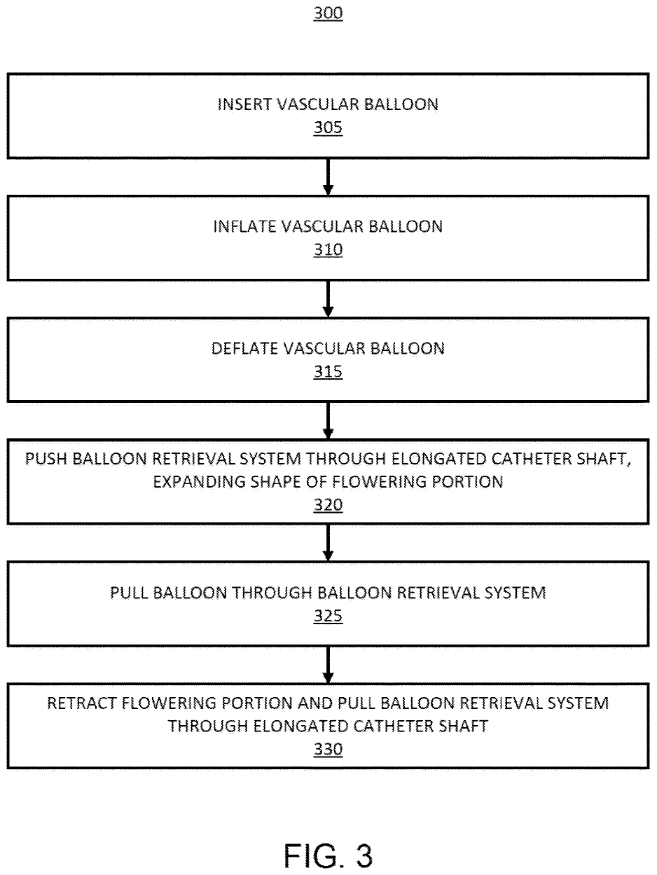

Referring now to FIG. 3, an example method 300 of using a balloon catheter system (e.g., balloon catheter system 100 of FIGS. 1-2) during a heart procedure is illustratively depicted, in accordance with various embodiments of the present disclosure.

At 305, a vascular balloon may be inserted (e.g., inserted into a heart, valve, artery, vein, etc.) through a balloon catheter system. According to various embodiments, the balloon may be inserted through a balloon retrieval system, positioned within an elongated catheter shaft of the balloon catheter system, to a position exterior to a distal end of the elongated catheter shaft. According to various embodiments, a guidewire may be inserted, followed by the balloon retrieval system, followed by the balloon. However, it is noted that the order of steps may be different, while maintaining the spirit and functionality of the present disclosure.

At 310, the balloon may be inflated, exterior to the distal end of the elongated catheter shaft. According to various embodiments, the balloon catheter system may comprise a balloon system which may comprise the balloon, a balloon inflation lumen, and a balloon inflation port configured to supply at least one inflation medium through the balloon inflation lumen to the balloon. According to various embodiments, inflating the balloon may comprise supplying the at least one inflation medium through the balloon inflation lumen to the balloon.

At 315, the balloon may be deflated. According to various embodiments, the balloon may be deflated through the balloon inflation lumen and/or through other suitable means.

According to various embodiments, the balloon retrieval system may comprise a sheath portion and a flowering portion. At 320, the balloon retrieval system may be pushed through the elongated catheter shaft, causing the flowering portion to extend past the distal end of the elongated catheter shaft, causing the flowering portion to expand in shape, forming a funnel which opens distally from the distal end of the elongated catheter shaft. According to various embodiments, this funneling may be configured to enable the flowering portion to enable safe, reliable withdrawal of larger bore vascular balloons into smaller, low profile, vascular sheaths.

At 325, the balloon, in a deflated configuration, may be pulled through the flowering portion and through the sheath portion, thereby pulling the balloon through the balloon retrieval systema and through the elongated catheter shaft.

According to various embodiments, at 330, after pulling the balloon, in the deflated configuration, through the flowering portion and through the sheath portion, the balloon retrieval system may be pulled through the elongated catheter shaft toward a proximal end of the elongated catheter shaft, causing the flowering portion to retract as the flowering portion is pulled through the elongated catheter shaft. According to various embodiments, the flowering portion may be retracted prior to the flowering portion being pulled through the elongated catheter shaft.

Methods of the present disclosure may be employed on any suitable vessel including, but not limited to, the inferior vena cava, the superior vena cava, the iliac veins, the aorta, the pulmonary artery, cardiac artery, or the pulmonary vein.

In this document, when terms such “first” and “second” are used to modify a noun, such use is simply intended to distinguish one item from another, and is not intended to require a sequential order unless specifically stated. The term “approximately,” when used in connection with a numeric value, is intended to include values that are close to, but not exactly, the number. For example, in some embodiments, the term “approximately” may include values that are within +/−10 percent of the value.

When used in this document, terms such as “top” and “bottom,” “upper” and “lower,” or “front” and “rear,” are not intended to have absolute orientations but are instead intended to describe relative positions of various components with respect to each other. For example, a first component may be an “upper” component and a second component may be a “lower” component when a device of which the components are a part is oriented in a first direction. The relative orientations of the components may be reversed, or the components may be on the same plane, if the orientation of the structure that contains the components is changed. The claims are intended to include all orientations of a device containing such components.

The features and functions described above, as well as alternatives, may be combined into many other different systems or applications. Various alternatives, modifications, variations or improvements may be made by those skilled in the art, each of which is also intended to be encompassed by the disclosed embodiments.

It will be apparent to one of ordinary skill in the art that various combinations and/or modifications and variations can be made in the perfusion dilatation catheter systems and devices of the present disclosure depending upon the specific needs for operation and as dictated by the therapeutic needs of the patient. Moreover, features illustrated or described as being part of one embodiment may be used on another embodiment to yield a still further embodiment.

1. A balloon catheter system, comprising:
an elongated catheter shaft:
having a proximal end and a distal end; and
enclosing:
a guidewire;
a balloon system; and
a balloon retrieval system;
the guidewire;
the balloon system, comprising a balloon positioned along the guidewire; and
the balloon retrieval system, comprising:
a flowering portion configured to expand from the distal end of the elongated catheter shaft; and
a sheath portion positioned within the elongated catheter shaft,
wherein:
the flowering portion is configured to funnel the balloon, in a deflated configuration, into the sheath portion, and
the sheath portion is configured to enable the balloon, in the deflated configuration, to be pulled through a central cavity therethrough.

2. The balloon catheter system of claim 1, wherein the elongated catheter shaft is a 14 Fr catheter shaft.

3. The balloon catheter system of claim 1, wherein the elongated catheter shaft encloses a plurality of lumens, comprising:
the flowering portion and the sheath portion of the balloon retrieval system; and
a balloon inflation lumen configured to inflate and deflate the balloon.

4. The balloon catheter system of claim 3, wherein:
the balloon system comprises the balloon inflation lumen, and
the balloon is configured to be inflated exterior to the distal end of the elongated catheter shaft.

5. The balloon catheter system of claim 4, wherein the sheath portion is configured to carry the balloon through the elongated catheter shaft prior to inflation of the balloon.

6. The balloon catheter system of claim 4, wherein the balloon system comprises a balloon inflation port configured to supply at least one inflation medium through the balloon inflation lumen to the balloon.

7. The balloon catheter system of claim 1, wherein the flowering portion is configured to retract after expansion in order to be pulled through the elongated catheter shaft.

8. The balloon catheter system of claim 1, wherein the sheath portion has a diameter of 10-15 Fr.

9. A method of retrieving a catheter balloon, comprising:
inserting a balloon through a balloon catheter system, wherein:
the balloon catheter system comprises:
an elongated catheter shaft:
having a proximal end and a distal end;
enclosing:
a guidewire;
a balloon system; and
a balloon retrieval system;
the guidewire;
the balloon system, comprising a balloon positioned along the guidewire; and
the balloon retrieval system, comprising:
a flowering portion configured to expand from the distal end of the elongated catheter shaft; and
a sheath portion positioned within the elongated catheter shaft, wherein:
the flowering portion is configured to funnel the balloon, in a deflated configuration, into the sheath portion, and
the sheath portion is configured to enable the balloon, in the deflated configuration, to be pulled through a central cavity therethrough, and
inserting the balloon through the balloon catheter system comprises inserting the balloon through the sheath portion beyond the distal portion of the elongated catheter shaft;
inflating the balloon exterior to the distal end of the elongated catheter shaft;
deflating the balloon;
expanding the flowering portion; and
pulling the balloon, in a deflated configuration, through the flowering portion and through the sheath portion, thereby pulling the balloon through the elongated catheter shaft.

10. The method of claim 9, wherein the elongated catheter shaft is a 14 Fr catheter shaft.

11. The method of claim 9, wherein:
the elongated catheter shaft encloses a plurality of lumens, comprising:
the flowering portion and the sheath portion of the balloon retrieval system; and
a balloon inflation lumen configured to inflate and deflate the balloon.

12. The method of claim 11, wherein:
the balloon system comprises:
the balloon inflation lumen; and
a balloon inflation port configured to supply at least one inflation medium through
the balloon inflation lumen to the balloon; and
inflating the balloon comprises supplying the at least one inflation medium through the balloon inflation lumen to the balloon.

13. The method of claim 9, further comprising, after pulling the balloon, in the deflated configuration, through the flowering portion and through the sheath portion:
retracting the flowering portion; and
pulling the flowering portion through the elongated catheter shaft.

14. The method of claim 9, wherein the sheath portion has a diameter of 10-15 Fr.
